(19) United States
(12) Patent Application Publication
(10) Pub. No.: US 2026/0216377 A1
(43) Pub. Date: Jul. 30, 2026

(54) Use of nuclear membrane rupture for transduction with recombinant AAV gene therapy vectors.

(71) Applicant: peter ward, New York, NY (US)

(72) Inventor: peter ward, New York, NY (US)

(21) Appl. No.: 19/574,110

(22) Filed: Mar. 20, 2026

(51) Int. Cl. A61K 48/00; C07K 16/10; C07K 16/18; C12N 15/86

(52) U.S. Cl. CPC A61K 48/005; A61K 48/0083; C07K 16/10; C07K 16/18; C12N 15/86; C07K 2317/569; C12N 2750/14143; C12N 2750/14145; C12N 2750/14151

(57) ABSTRACT

A method for transducing cells with rAAV vectors that results in more rapid transduction than standard infection is described. The method involves induced nuclear membrane rupture. It requires less vector to achieve the same transduction level as standard infection and transduces individual cells with multiple vectors, even at low percentages of total transduced cells. It may be useful in treating people bitten by venomous snakes or who have been exposed to rabies. In one iteration it can target cells that have endogenous rupture of their nuclear membranes.

### FIELD OF THE INVENTION

Method of infection with rAAV vectors that provides faster transduction and transduction targeted to cells with ruptured nuclear membranes that may be useful for treating patients bitten by rabid animals or venomous snakes or patients with conditions characterized by nuclear membrane rupture.

### BACKGROUND

A gene therapy method that can produce a therapeutic protein more quickly than present protocols would in some cases be useful. Cases for this need for rapid in vivo protein production would prominently include infectious disease in which the protein to be encoded by the delivered transgene is an antigen or an immunoglobulin. A second need is for immunoglobulin type molecules (immunoadhesins) to neutralize protein poisons such as snake venoms. A possible method for rapid introduction can be based on gene therapy with adeno-associated virus vectors. The genes carried by the vector could code for a potential antigen or alternatively for an antibody or nano body pre-selected to be directed against the infectious agent. While immunoglobulin injected into the patient would be the most rapid way to introduce a therapeutic antibody into a patient, immunoglobulins are expensive, often unavailable locally, and frequently have unwanted side effects due to foreign material. In some localities, in which infrastructure and transportation are non-optimal, the timely delivery of immunoglobulins to the patient is difficult. Gene therapy with a transgene that codes for an antibody or nanobody is slower than administration of immunoglobulin. However, it provides a faster therapeutic response than vaccination and avoids some of the problems associated with immunoglobulin usage.

A limitation to the use of AAV-based vectors as a vehicle for rapid gene delivery is the time between infection of the target cell and production of the transgene encoded product. Described herein is a method that enables faster expression of the transgene; it also results in multiple expressing vector genomes in transduced cells.

### SUMMARY

As stated in the Background section there is an unmet need in resource poor regions to treat patients who have been bitten by poisonous snakes or by rabid dogs. There are treatments for these events but there is a failure in many cases to use them. Most of this failure is due to the remoteness of the event and the expense of the treatment. Gene therapy with rAAV vectors might be able to surmount these issues by widespread and more local storage of these vector and by a lower cost for the treatment.

A potential blockage to the usefulness of this approach however is the time between administration of the vector and an immune response in the patients. Technologies exist to overcome some parts of this problem, namely the construction of vectors which code not for an antigen but rather for a predetermined antibody. A further refinement is coding for nanobodies rather than antibodies. A remaining issue is the length of time between administration of the vector to the patient and the manufacture of the encoded protein e.g. an antibody or nanobody. After infection rAAV vectors enter cells and are transported to the nuclear region quickly. The inefficiency maps to the time interval between localization of the vector near the nucleus and transcription.

Described herein is the use of physical manipulation of the patient's cells to effect rapid access of the genome contained within the vector to the protein synthesis apparatus of the patient's cells. This rapid access is accomplished by squeezing the cells to an extent such that the nuclear membrane is ruptured but the cellular membrane is not. This leads to measurably more rapid translation. The vector in this procedure can enter the nucleus in a passive manner apparently overcoming the barrier to nuclear entry seen in standard infection protocols. Moreover, this passive mechanism permits entry of particles that seem unable to gain entrance to the nucleus by standard infection. This permits the use of less vector. (In standard infection protocols MOIs of hundreds to thousands of rAAV vector particles are required to transduce a cell.). This method also results in multiple vectors often entering the same cell. This multiple entry, not seen in normal infection unless very high levels of vector are given, results in higher expression levels of the transgene encoded protein. Described herein is a protocol for rapid transduction of suspended cells which could be utilized with a patient's blood.

This method of vector administration also has advantages for gene therapy more generally, i.e. not just for snake venom, and infectious diseases or other events in which rapid expression of the transgene is important. It also allows for vectors of different genotype to enter the same cell. A limitation in the use of rAAV vectors is their small packaging capacity. Multiple vectors that encode different transgenes will, if present in the same cell, permit recombination that will allow the production of full-length genomes for larger genes. A limitation particular to the methodology described herein for correcting genetic diseases is that the targeted cells must be single cell suspensions; therefore administration will in one iteration necessarily be ex vivo.

An advantage particular to this method is that when Rep-dependent integration of the rAAV genome into the privileged AAVS1 site is desired, coinfection of a wt AAV into the same cells as a rAAV is required. This is hard to achieve without an infection level that puts vector and wt AAV together into most cells. In this method however the entrance of vectors into the nucleus are not independent single-cell events, but rather events in which multiple particles enter together making it likely that in coinfections, even when low numbers of cells acquire the rAAV vector, many of those cells will simultaneously acquires a wt AAV virus.

As said, if used in vivo, there is little opportunity to squeeze the target cells. However, there are endogenous mechanisms that cause cell squeezing. These mechanisms provides an rAAV vector targeting signal for cells that experience nuclear membrane rupture from endogenous cell squeezing, e.g. metastasizing cancer cells, cancer cells more generally, cells from individuals with any of the many conditions associated with mutations in the nuclear lamina. Vectors can be used that have in themselves no specific cell-targeting sequences or targeting mechanism. Transduction will be targeted to cells that have a nuclear membrane-rupture defect.

In all these cases the method can take advantage of the need for much lower amounts of vector since entrance of vector into nucleus can, because it is passive, be accomplished by vector that is not optimally constructed. Transduction in which nuclear membrane breakage is endogenous rather than induced will require longer times between administration of the vector and production of the transgene encoded protein. The advantage, due to infection with lower amounts of vector, will be a reduced adaptive immune response, with the goal of reduction to a level of vector administration that will more readily permit readministration of the vector.

### DESCRIPTION

The description is organized as follows: Brief description of figures. Introduction, glossary, narrative of basic AAV biology as needed to understand application, standard protocols and methods and materials used in the assays of this application, new methods, results, uses.

### BRIEF DESCRIPTION OF FIGURES AND TABLES

FIG. 1

A schematic showing location of rAAV vector particles within an infected cell. Particles remain clustered outside the nucleus. Cyt.; cytoplasm. Nuc.: nucleus.

FIG. 2

A schematic showing location of the lamina network in relation to the outer nuclear membrane (OM) and the inner nuclear membrane (IM). Cyt.; cytoplasm. Nuc.: nucleus.

FIG. 3

A schematic showing the first stages of a cell traversing an opening much narrower than the cell. As seen the nucleus is extensively deformed with a bleb emerging from the main body of the nucleus. Cyt.; cytoplasm. Nuc.: nucleus.

FIG. 4

Schematic that is a continuation of the schematic of FIG. 3. The nuclear membrane has ruptured at the bleb and nuclear contents are flowing into the cytoplasm. Not shown, for the sake of simplicity, is the accompanying process of cytoplasmic components passing into the nucleus. Cyt.; cytoplasm. Nuc.: nucleus.

FIG. 5

Schematic showing the mechanism of squeezing cells. Trapezoidal structure represents tip of pipet containing cells suspended in media. Pipet tip is in contact with the surface of a glass slide. This contact is shown to be at a more pronounced angle than in actual practice to illustrate the principle. Media and cells are squeezed out of the pipet then drawn back into the pipet tip repeatedly. The two headed arrow represents the dimension of the hypothetical gap between the rim of the pipet tip and the glass slide at which, when the cells are squeezed through, the nuclear membrane will rupture but the cell membrane will not. This mechanism is particular to this application and is meant only to illustrate the general principle.

FIG. 6

Micrographs of two plates of confluent Hep G2 cells that were infected with the GFP expressing rAAV vector AAV2-TRUF11 six days previously. The plate on the right was plated with cells squeezed after infection but before plating. The plate on the left was plated with cells that had not been squeezed. Photograph was taken with UV light at a reduced exposure so that only the cells with the high amounts of GFP expression are visible. At a lower exposure many cells on the left plate show GFP expression.

FIG. 7

Graphs of the results of a a flow cytometry analysis of 3 plates of AAV2-TRUF11 infected cells. Shown is a graph of only the GFP positive cells from that flow cytometry. The K and L plates were plated with cells that had been passed through a flow cytometry filter multiple times after infection as a control. The M plate was plated with cells that were squeezed as described. Harvest and flow cytometry were performed three days post infection. The dot plots were measured for the number of cells expressing GFP and for the mean intensity of that expression, with the results from the K plate normalized to 1.0. The table shows a comparison of these values for the K and M plate. The relative number of positive cells and the brightness of the cells were multiplied to give the relative amount of expression on the two plates, designated by “X”. With the M plate having 4.3 fold more expression than the K plate. The graph has more cells on the right. This is consistent with more of the GFP positive cells having higher expression.

FIG. 8

Flow cytometry analysis of three infections. Shown on the left are dot plots from the three plates and on the right a graph of the positive cells from each dot plot. The cells on the the N plate were not squeezed. The cells plated on the O and S plates were squeezed before plating. All 3 plates were infected with the same amount of AAV2-TRUF11 vector. The vector used to infect the S plate also contained some AAV-mCherry vector. The mCherry vector does not give a detectable fluoresce with the filter used to measure GFP fluorescence.

Table 1

The data of the assay of FIG. 8 in numerical form. The relative number of GFP expressing cells and the mean expression levels of the positive cells are measured with the control plate, Plate N, being normalized to 1.0 as with the data from plates K and M.

Table 2

Data from the assay of FIG. 8. The number of GFP positive cells in each bin are shown. P2 is the bin with the highest expressing cells, i.e. the brightest. P3 the next brigtest etc. Higher expression in a cell is most likely due to more GFP expressing genomes in that cell. The bin size is chosen so that every 3 bins is a 10-fold increase in expression.

FIGS. 9, 10, 11

Three sets of micrographs of cells coinfected with vector that is a mixture of AAV2-TRUF11 and AAV2-mcherry were photographed under UV. The two fields of view in each figure are the same. The left panel is photographed with a green filter. The right panel is photographed through a red filter. As discussed, the GFP and mCherry fluorescence do not overlap sufficiently to be detected under this amount of illumination and with these filters. The population has fewer cells with fluorescence from the AAV-mCherry vector than from the AAV2-TRUF11 vector. The panels are all with confluent populations, when photographed, showing a low percentage of fluorescing cells. Most cells containing fluorescing mCherry genomes also contain fluorescing GFP genomes. Blow each micrograph is a duplicate with several cells that are fluorescing under both the red and green filers numbered. Not all such co fluorescing cells were numbered.

FIG. 12

A potential delivery device for treating snakebite patients and patients who may have been exposed to rabies. Patient's blood is withdrawn. Blood is either used as is or given low speed centrifugation and only the fraction containing while blood cell is retained. Blood is infected with vector. Short incubation. Blood is transferred to a syringe-like structure as shown. Blood is given back to patient after being forced through a membrane or matrix near the tip of this syringe-like device. The pores are of a size to rupture nuclear membranes without rupturing cell membranes. Blood can be withdrawn from patient using the same syringe. In the case of using one syringe for both withdrawal and redelivery, top part of syringe must be replaced. For withdrawal of blood the top part contains no membrane or matrix. After infection and incubation top part is removed and replaced with a top part that has the membrane/matrix.

### INTRODUCTION TO DESCRIPTION

A limitation in transduction by AAV vectors is a failure of the vector carried transgene to be delivered into the nucleus of the target cell in a timely manner. The mechanisms between rAAV infection of the cell and this delivery are not fully understood; it is likely that there are multiple pathways. Developing a complete picture of these mechanisms is hampered by differences in experimental systems between different groups, e.g. the use of recombinant AAV vectors vs wt AAV, different AAV serotypes, and different target cells.

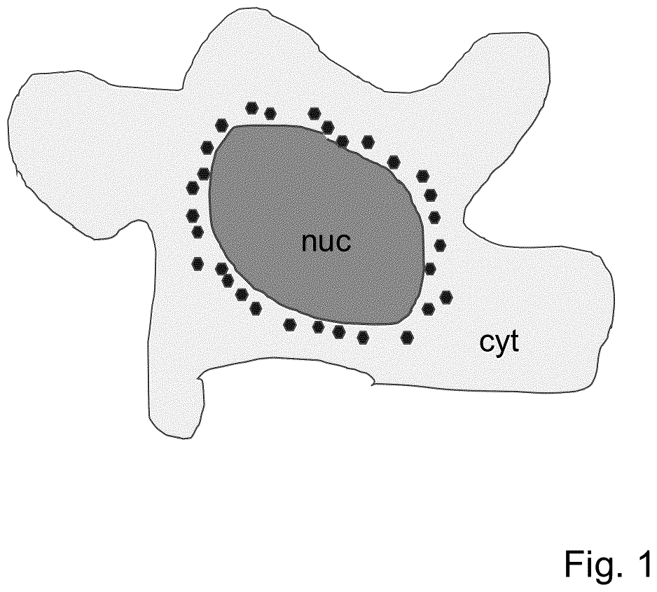

Some general statements can be made however: Upon infection, AAV-based vectors migrate to the nuclear membrane but remain clustered in this region without a rapid accompanying entrance into the nucleus, FIG. 1. The precise mechanism of this transport are not established in detail, but the transport to the nucleus happens rather quickly (1). In one report aav particle were transported to the nuclear membrane in a microtubule supported unidirectional manner in 5-10 sec. (2).

Almost all particles remain outside the nucleus. (3) It is unknown if there is some property of the particles that determines whether a particular particle is imported into the nucleus or remains extranuclear. It is still a point of contention whether the genome of the AAV or rAAV particle enters the nucleus without the capsid or whether the intact particle is imported into the nucleus and the particle's genome is then released. A third possibility is that the particle's genome is released as the particle is imported into the nucleus. Data has been reported that implies that capsid uncoating occurs before or during entry and few intact capsids are found in the nucleus. This work was performed with wtAAV rather than with rAAV vector. It was also observed that nuclear entry was different depending whether infection was done with or without an adenovirus coinfection. (4) Another report also finds that in the absence of helper virus little intact capsid is seen in the nucleus, whereas viral genomes were detectable in the nucleus. (5).

Alternatively it has been reported that intact capsids of wt AAV2 persisted in the nucleus for weeks after infection. These workers described less persistence for capsid from serotypes other than AAV2. They also suggest that uncoating is a rate limiting step on the path to transduction. (6)

Other data has supported entry of an intact or relatively intact particle. (3). (7) The latter group also showed that microinjection into the nucleus of capsid specific antibodies blocked infection. This is probably the strongest evidence that an intact capsid in the nucleus is a step on the way to productive infection or transduction.

More recent data has been presented that is consistent with the particle entering the nucleus through the nuclear pore complex. (8) (9). This last study argues that entry of the particle into the nucleus is a limiting step in rAAV transduction.

In the autonomous parvovirus, Minute virus of mice, it has been reported that the viral capsid can ruptures the nuclear membrane suggesting a possible mechanism for viral entry. (10) (11).

While it remains an unresolved question at what point uncoating occurs, i.e before, during, or after import of the vector's genome into the target nucleus, it seems established that uncoating is a gate keeping step on the road to transduction. This point has been more recently highlighted in experiments by the Salvetti and Buning labs, who, using a peptide display library and dendritic cell infection showed a correlation between efficient uncoating and efficient transduction. (12)

Entry of AAV and rAAV particles into the cell and migration of these particles seems to be a universal capacity of these particles. The particles are able to enter most cell types and are then carried to the periphery of the nucleus. The great majority of the rAAV particles, however, never enter the nucleus. The contrast between the high infection efficiency of the wt AAV and the transduction inefficiency of rAAV suggests that present production methods for rAAV produce viral particles that are less well made than those of the wt AAV (13) and that one consequence of this is particles that are deficient in nuclear entry.

This inability to efficiently enter the nucleus is a roadblock for productive transduction. The method of this application, since it relies on the simple presence of a vector particle at the right place and not on the ability of the particle to navigate its way through the nuclear membrane, bypasses this putative blockage.

### Glossary

Transgene: Herein transgene refers to the genetic cargo carried within the recombinant adeno-associated viral vector.

Immunoadhesins: Molecules with binding motifs as found in an antibody. Herein all immunoadhesins are antibodies or nanobodies.

This application describes a simple method, applicable in certain circumstances, to rapidly bypass some of the blockage occurring at the nuclear membrane with rAAV vectors thereby permitting a more rapid expression of the transgene carried by the vector. It also describes that without induced nuclear membrane breakage, if cells suffer from endogenous nuclear membrane breakage, they can be transduced, albeit slowly, with low levels of vector particles because particles can enter the nucleus in a passive manner,

Standard Methods for Making and Using rAAV Vectors that are as Used in this Application:

Virus production was by the standard rAAV production method (14). Recombinant virus, AAV2-TRUF11, was produced by transfection of equimolar amounts of plasmids: pAAV2-TRUF11, pXX6, and an AAV2 Rep-Cap construct into non-confluent HEK 293T cells. pAAV2-TRUF11 (15) contains an enhanced green fluorescent protein, GFP. It also contains a Neomycin resistance gene, however, at no time during these assays were cells treated with G418.). pXX6 contains adenovirus genes necessary for AAV replication. Transfection was mediated by CaCl2.

Detailed Transfection Protocol for Making Recombinant Adeno-Associated Virus (rAAV) Vectors: This is a Standard Protocol for Producing rAAV Vectors:
HEK 293T cells were plated in DMEM with 10% Fetal Bovine Serum (FBS) and allowed to grow until the plate was from 40-60% confluent, generally overnight.
Media was replaced with low glucose DMEM with 2% FBS.
Plates were incubated for 2 hours in this media, then the transfection mix was added.
The three plasmids (pAAV2-TRUF 11, pXX680, and pXX2) were combined in one tube at amounts that are approximately equimolar. The plasmids are approximately the same size, therefore equal amount by mass can be used; e.g. approximately 0.8 μg per well of each plasmid in one well of a 6-well plate. One well of a 6-well plate holds typically 2.0 mls of media. The protocol can be scaled up or down with the same relative amount of materials.)
Add water to plasmid mixture to bring volume to 15 μl
Mix
10.0 μl CaCl₂ (2.5M)
Mix
75 μl sdH2O. i.e. bring up to 100 μl
100 μl HeBS. Add slowly drop by drop with continuous swirling

### HeBS Composition:

50 mM Hepes
280 mM Na Cl
1.5 mM Na₂HPO₄
(pH adjusted to 7.1 with NaOh)
Bubble air through mix at least 20×.
Let mixture sit before adding to cells. 15′ (this is slightly longer than is usually suggested)
Mix mixture before putting on cells
Add to cells slowly drop by drop with gentle swirling of the plate
Harvest at 68-72 hrs. post transfection.
Dislodge cells by squirting with media
Spin 4 min at moderate speed e.g. 7500 RPM
Remove supernatant.
Resuspend pellet in 150 μl TE
Freeze/thaw 3×
Pipet a few times after each thaw to partially disaggregate clumps.
After 3^{rd} thaw, added:
6.0 μl 3.0 M Nacl
4.0 μl 100 mM MgCl
0.8 μl 1.0 M tris pH. 7.8
2.0 μl benzonase at 1 unit/ul. No DNAse
37 degrees for 10 min
Remove and use pipettman to completely disperse lumps
37 degrees for 4 hours
Store at −80 degrees.
   Infection of Target Cells with Vector:

The cells used as a model target were Hep G2 cells. (Hep G2 is a standard cell line isolated from a human hepatocellular carcinoma) They were grown in DMEM in 24-well plates supplemented with 10% fetal bovine serum. Media was changed immediately prior to infection. Upon infection cells were maintained in DMEM and 10% FBS with Penicillin (50 units/ml) and Streptomycin (50 μg/ml). Cells were infected at approximately 30% confluence. The vectors are simply added to the cells.

In the sections of this application in which nuclear membrane rupture was induced the infection protocol is slightly altered. The hep G2 cells were infected prior to plating. Then after an incubation period they were forced through a tight gap. In this application this was accomplished by pipetting the suspended cells in an out of a pipet tip in contact with a glass slide (as described more fully below). Then cells were plated and thereafter treated as a normally infected population.

Recombinant adeno-associated virus (rAAV) vectors are derived from the virus AAV and possess an AAV capsid. AAV or adeno-associated virus as used in this application is understood to refer to all the natural and modified parvoviruses that are being used or will in the future be used to perform AAV-like functions to include but not be limited to the following: The 12 originally described serotypes of AAV as well as numerous other isolates with differences in their capsid or genomic sequences. In this application the AAV capsid used for the recombinant vector is AAV2. However, AAV is meant to stand in for all the other AAVs and mutant AAVs, both natural and constructed in the laboratory, or parvovirus chimera, as well as other parvoviruses. (AAV is a member of the family parvoviridae.) These include but are not limited to: the known canonical AAV serotypes: AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), as well as modified AAVs and non-human AAVs; AAVbb2, AAVcy5, AAVrh10, AAVrh20, AAVrh39, AAVrh43, AAVrh64R1, AAVhu37avian AAV, bovine AAV, canine AAV, equine AAV, primate AAV, non-primate AAV, ovine AAV, as well as AAVs comprising a capsid protein of one AAV subtype and genomic material of another subtype, and AAVs with mutant or chemically modified capsid protein, or AAVs in which the capsid is chimeric i.e. an AAV capsid with regions derived from more than one AAV serotype or other parvoviruses, and the related, but non-AAV parvoviruses or sequences from these parvoviruses, e.g. bocaviruses, goose parvovirus, and others. Also included as well are those that will be developed in the future including additionally those parvoviruses that have been modified in the laboratory to alter the properties of the virus.

The figures consist of Dot plots from a flow cytometry or graphs based on that flow cytometry data.

Cells were processed for flow cytometry on a Becton Dickinson Canto flow cytometer using FACS Diva software. Cells were determined to be positive for GFP expression by FITC/PE ratio as described previously (16). When plotting FITC vs. PE in flow cytometry, cells whose greenness is derived from endogenous sources have a defined PE/FITC ratio when graphed. This ratio is different for greenness derived from the GFP protein. This allows a definitive separation of cells expressing FITC from those not expressing FITC. In all the assays shown herein the cells were first gated on a flow cytometry of PE/FITC, then color marked as having green from FITC or not. On graphs, of this application in which the cytometer is plotting counts vs FITC, the Y-axis (i.e. the counts axis) was showing the numbers of cells at various levels of FITC expression. The x-axis is showing the level of FITC expression. On all the dot plots of this application FITC is plotted against SSC. In these the events/cells that show up as fainter dots, i.e. primarily to the right are GFP expressing. The cells on the left, i.e. the darker cells in the very dense cluster, are not GFP expressing. In the colored version the red cells, i.e. the cells to the right of the dense cluster, express GFP; the blue cells (i.e. the dense cluster) do not. The cells in the SSC vs FITC dot plots had been first gated by PE vs FITC to distinguish the GFP expressing cells from non-GFP expressing cells. The GFP expressing cells were color coded red. The non GFP expressing cells were coded blue. In the non-colored version the blue cells are dark. The red cells are gray.

Novel Methods Found in this Application.

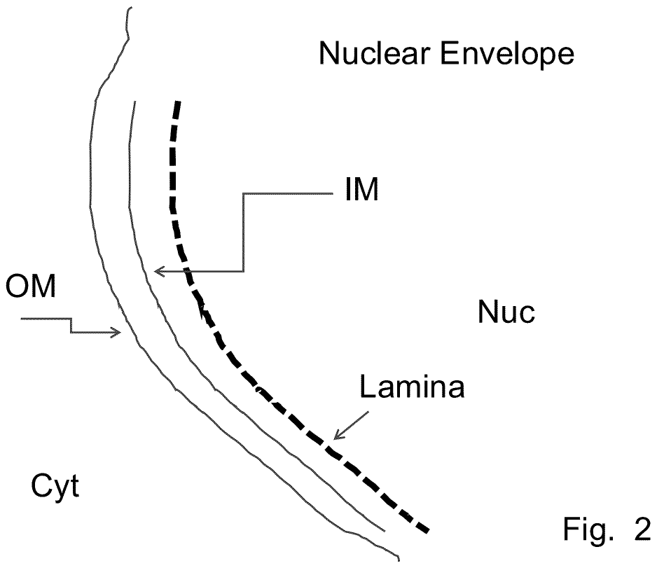

Somewhat surprisingly the cellular membrane is more flexible than the nuclear membrane. This rigidity of the nuclear membrane is due to a lamina that lies inside both the outer and inner nuclear membrane, FIG. 2. While a significant breakage of the cellular membrane results in cell death, breakage of the nuclear membrane does not as long as the break is repaired (17). This raises the possibility that mechanical rupture of the nuclear membrane without extensively damaging the cell membrane might facilitate expression of the vector genome by allowing vector to enter the nucleus in a passive manner, thereby bypassing the blockage at the nuclear membrane. This entrance into the nucleus is made possible by the clustering of vector particles around the nucleus.

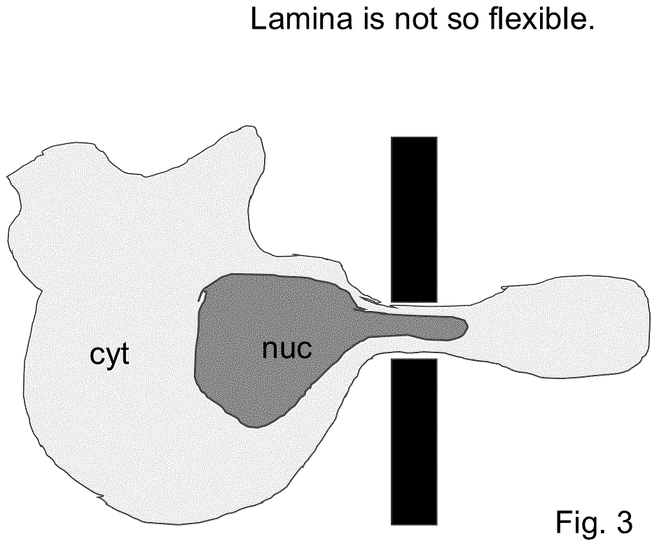

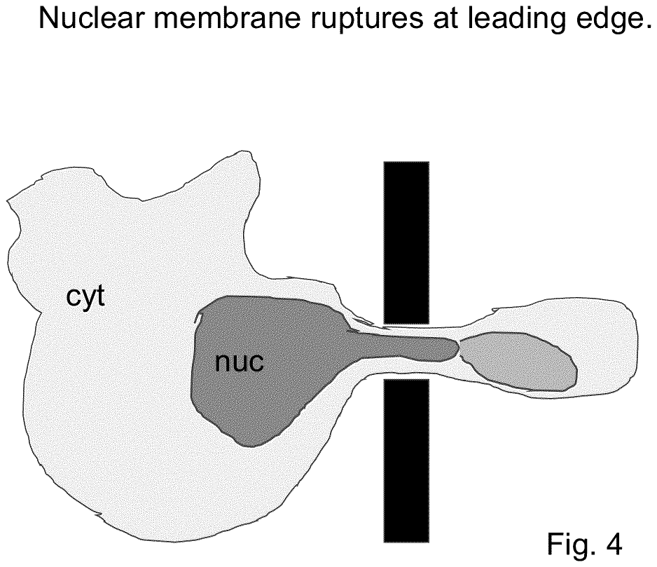

This application shows that it is possible to disturb the cell mechanically in such a manner as to permit a more rapid entrance of a vector particle or multiple particles into the nucleus while the cellular membrane is not breached. The survival of the cell is demonstrated by its ability to produce the transgene. FIG. 3 and FIG. 4 are cartoons illustrating how a cell can be squeezed through a small opening with consequent rupture of the nuclear membrane without rupture of the cellular membrane.

To test this a simple but crude method of squeezing cells was improvised. (to use this method in a real application cells would be forced cells to pass through a membrane or matrix with pores of the approximately correct dimensions. The membrane or matrix should be of sufficient thickness so that each cell will pass through multiple pores some of which are sufficiently small to accomplish breakage.). In this improvised example Hep G2 cells were plated on standard cell culture plates in standard media and allowed to grow until confluent. The cells were harvested then infected with the GFP expressing vector AAV2-TRUF11.

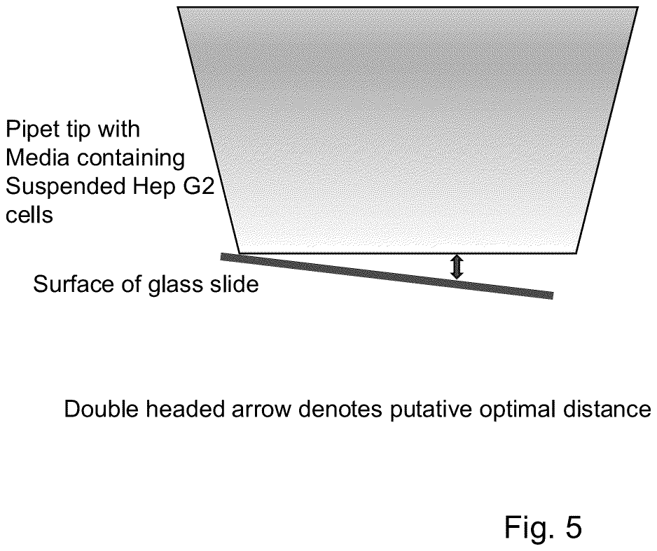

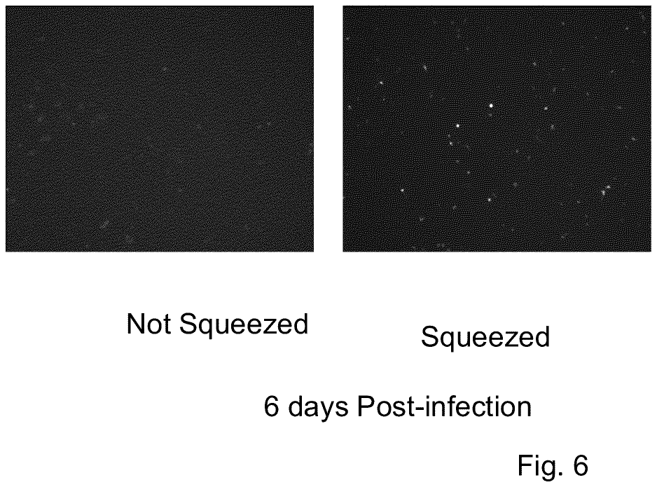

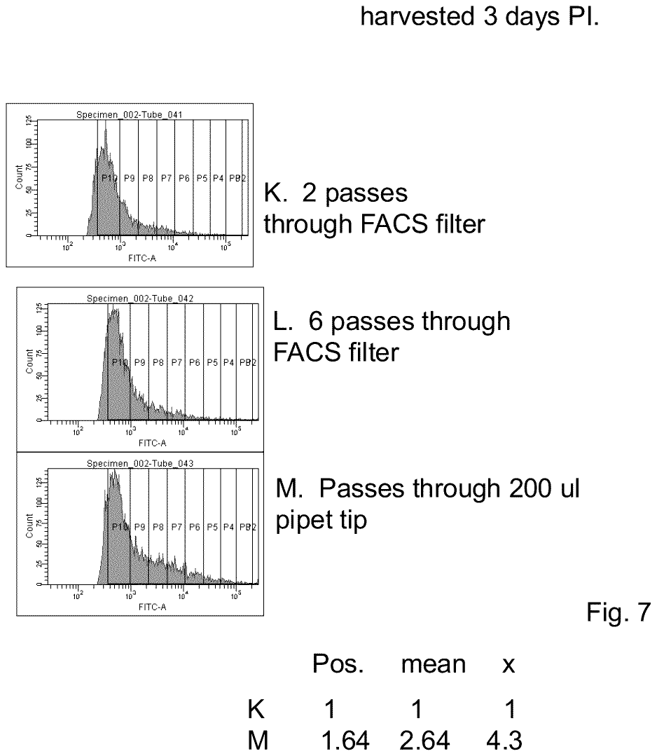

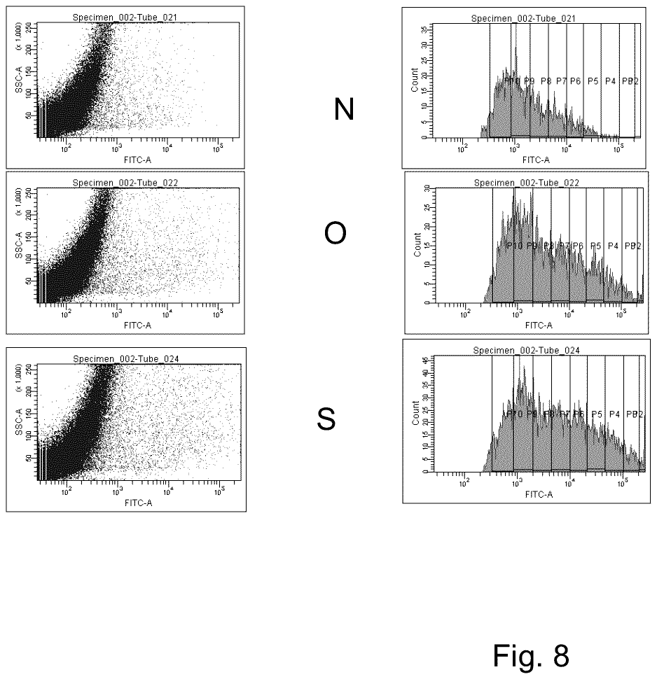

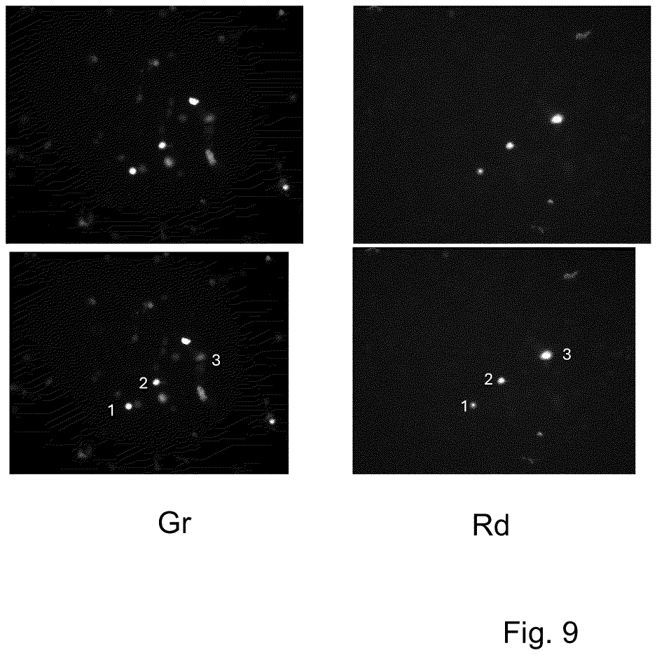

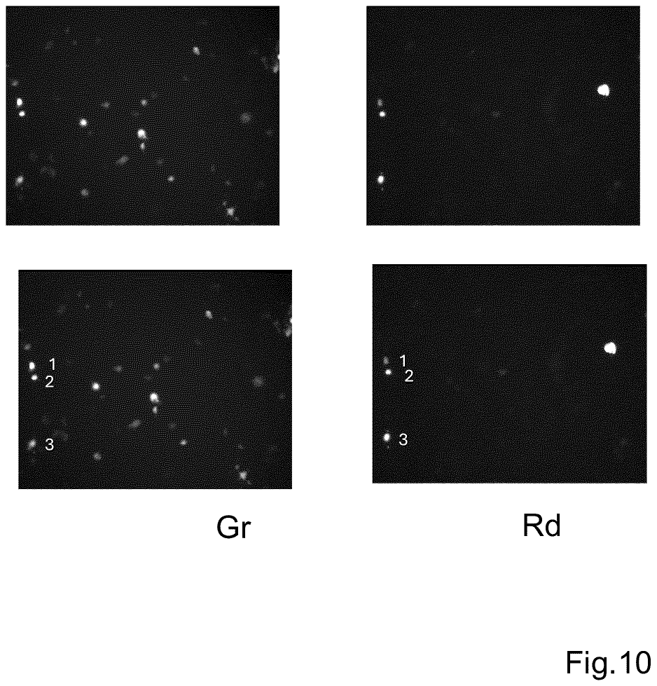

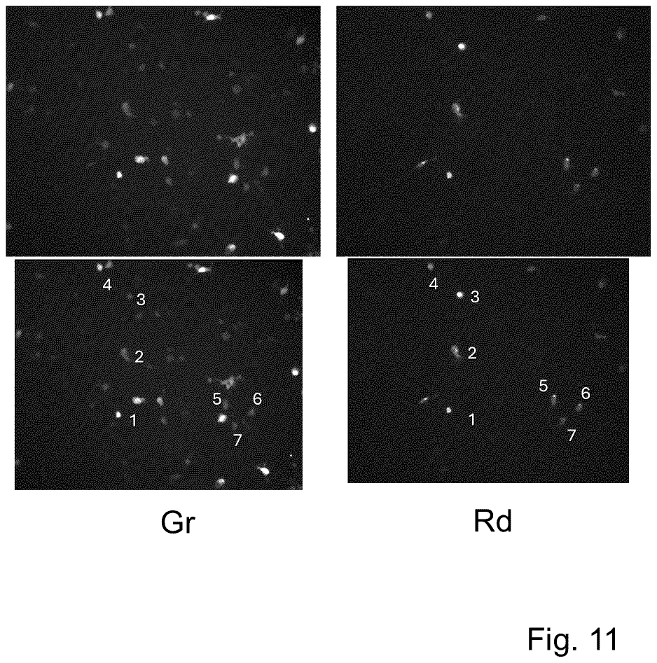

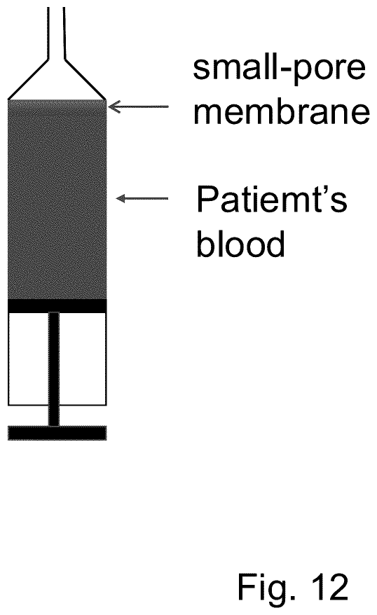

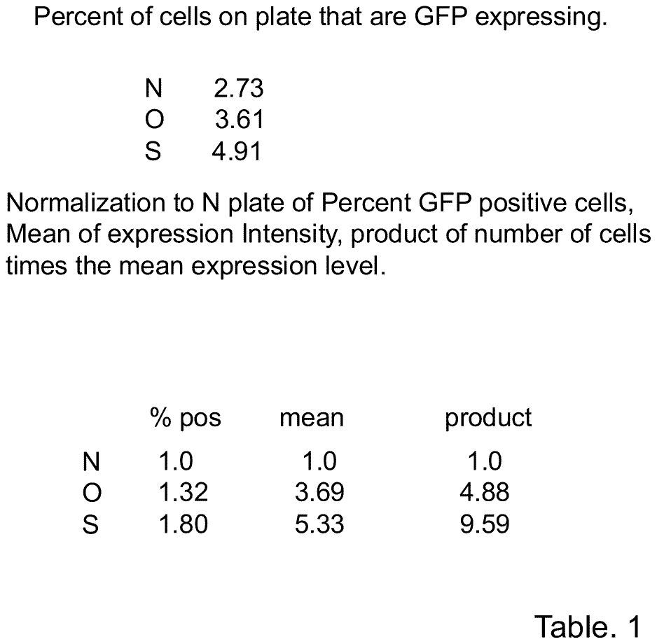

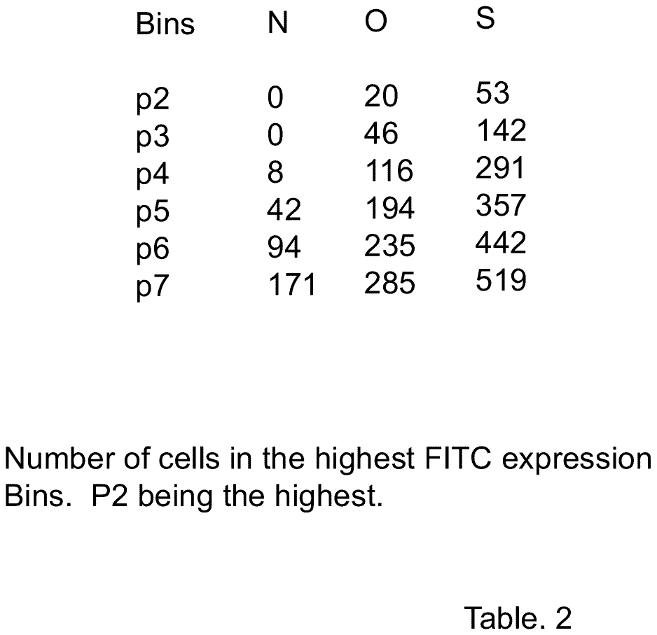

After a 4 hour incubation the suspended cells were pipetted up and down in a 200 μl pipettman using standard pipet tips. The pipet tip was pressed against a standard glass microscope slide. The media containing the vector was pushed out of the pipet tip onto the slide, this was followed by sucking the media, which contains the vector, back into the pipet. This was repeated 30 times. The pipet tip remained pressed against the glass slide for the whole procedure. Care was taken not to create air bubbles in the media. The cells were then plated as normal and allowed to grow in standard cell culture conditions. The expectation is that some small fraction of the cells, will, with each repetition of the pipetting, pass through a gap of the requisite dimensions so that the nuclear envelope is ruptured while the cell membrane is not. FIG. 5. Is a cartoon illustrating the mechanics of this. It shows the pressing of the pipet tip against a glass slide. The gap between the glass slide and the rim of the pipet tip will vary. The media containing the suspended cells is squeezed between the rim of the pipet tip and the surface of the glass slide. At some points the gap between pipet rim and slide is the appropriate size for rupture of the nuclear membrane without rupture of the cell membrane. The appropriate size of this gap will need to be determined for each type of target cell. It should be understood that because of the crude method of squeezing as employed here, most of the transduced cells in the data described below had vector that entered the nucleus without membrane breakage.

### Description of Results:

An aliquot of cells was infected with AAV2-TRUF11 vector. The infected cells were divided into two equal portions. One portion was plated as is while the other portion was squeezed as described above, then plated.

At 6 days post infection plates were photographed, then harvested. This was followed by analysis of the cells by flow cytometry. Photographs of the plates are shown in FIG. 6. Six days post infection is a length of time that permits Hep G2 cells infected with AAV2-TRUF11 vector to achieve maximum GFP expression. This exposure, which is a low exposure micrograph, shows many more brightly expressing cells on the right plate (which is the plate with squeezed cells) than on the left plate (which is the plate with non squeezed cells). At a higher exposure it can be seen that there are many GFP expressing cells on the non squeezed plate also (data not shown). At this low level of infection with the AAV2-TRUF11 virus, GFP positive cells with an expression level that is consistent with more than one expressing vector in the cell are infrequent when infection is done in the normal way. This is consistent with random infection at low levels of transduction. This the case even though essentially all of the infected cells will have vector particles clustered around the nucleus (3). As seen in subsequent figures and tables there are more GFP expressing cells on the plates with squeezed cells, but it is only a marginal increase in these assays. FIG. 6 shows that more of the squeezed cells are expressing visibly higher levels of GFP, which is consistent with more than one expressing vector in these cells.

As discussed in the references in the Introduction to this Description, the infecting rAAV particle would seem to reach the nucleus in times much shorter than the 4 hour incubation used in the assays described herein. The amount of time required for nuclear localization will need to be determined for different types of target cells and different AAV serotypes. In general the time needed is likely to be much less than 4 hours as demonstrated in the reports referenced in the Introduction section above.

A comparison of total transduction between squeezed and unsqueezed cells is shown. Hep G2 cells were infected as above. Cells were squeezed as described above then replated. The cells of plates K and L were passaged through a FACS filter which would snot be expected to squeeze the cells sufficiently to cause nuclear breakage. The cells of Plate M were squeezed by the repeated pipetting as described above.

FIG. 7 shows graphs of the FITC positive cells from flow cytometer dot plots from 3 days post infection. (That is the cells that did not express FITC on the original PE/FITC gating are not visible in this Figure. The PE/FITC gating is not shown.) As can be seen the graph shows a greater number of higher GFP expressing cells on Plate M, than on Plate K and Plate L. This is consistent with the presence of cells with more than one vector in some cells on the M plate as compared with the K and L plates. (Note that the FITC scale is a log scale.) A comparison of the main peaks in each graph, which are of approximately the same height, shows that the squeezing is not resulting in cell death. The table gives the values derived from this flow cytometry with the number of positive cells, the mean FITC expression from the positive cells, and the product of these two values being shown. The values for the K plate are normalized to 1.0. When the median expression on the two plates is multiplied by the number of cell on that plate, designated by ‘X’, it is seen that the M plate has over a 4-fold greater expression of the GFP protein than does the K plate. A comparison of the 1.64 increase to the 2.64 increase indicates that most of this 4-fold advantage is due to a greater number of cells with more than one vector on Plate M.

To show that expression of the transgene occurs more quickly in cells that are squeezed and to further demonstrate that it is likely that the presumed greater number of vectors in some cells on the plate with squeezed cells is due to more vector entering the nucleus of the positive cells the following assays are shown.

Three plates were infected and treated as follows:

| | | |
|---|---|---|
| N. AAV2-TRUF11. | Not squeezed | |
| O. AAV2-TRUF11. | Squeezed | |
| S. AAV2-TRUF11. | +AAV-mCherry. | Squeezed |

All Three plates were infected with AAV2-TRUF11. One plate, S, was also infected with AAV-m Cherry, which fluoresces red when visualized with the appropriate filter.

One cell aliquot was plated without squeezing, N. The other two aliquots, O and S, were squeezed by pipetting up and down before plating.

The plates were checked under UV light 18 hrs after plating. Plate N showed no evidence of GFP expression by visible inspection. (In numerous assays with this vector in Hep G2 cells this is the expected result for one day post infection.) In contrast Plates O and S showed readily visible expression.

FIG. 8 and Table 1 and Table 2 show the data from the N, O, and S plates after harvesting at six days pst-infection, comparing cells that were squeezed with cells that were not squeezed. FIG. 8 shows that only a small fraction of the cells on each plate are positive for GFP expression Seen in the FIG. 8 dot plots and graphs of the dot plots is that there are more GFP expressing cells on the plates containing the squeezed cells, i.e. plates O and S, than on the plate containing the non-squeezed cells, i.e. plate N. Additionally, the mean GFP expression level of the O and S plates is higher than the N plate. Notably, in comparing the O and S plates, the mean expression level is higher on the plate that has the most GFP expressing cells, the S plate. The conclusion from these last two points, is that the squeezing that is resulting in more cells with an expressing vector is also giving more cells with multiple expressing vectors and that it is the multiple expressing vectors in many cells that is the main component of the increased level of GFP expression on the O and S plates. This is the same conclusion drawn from a comparison of the K and L plates described above. (In hundreds of infections with the AAV2-TRUF11 vectors, when equivalent amounts of vectors from the same vector prep are compared there is little variation in the number of cells that become GFP expressing, even when the infections are performed months apart. Additionally at this low level of infection there were few cells expressing GFP, in the N plate, at a level consistent with more than one expressing vector in the cell.)

On the S plate 5% of the cells were positive for GFP expression. Under the red filter less than 1% of the cells were visibly expressing mCherry. It should be noted that there was no detectable overlap in the fluorescent signal between the two vectors. Plates infected only with AAV2-TRUF11 vector showed fluorescence under the filter for GFP expression. They showed no fluorescence under the filter for mCherry expression. The reverse was true for plates infected only with the mCherry vector. In previous coinfection experiments with these two vectors, i.e. AAV2-TRUF11 and mCherry it was exceedingly rare to observe a cell simultaneously expressing both the GFP of TRUF11 and the red of mCherry. Since cells in FIG. 8 are being measured for GFP expression alone, the larger number of higher expressing cells on The S plate cannot be from expression of the cells infected with mCherry. It is most likely due to a better result from the squeezing procedure, i.e. more of the cells in this aliquot had their nuclear membrane disrupted without disruption of the cellular membrane. This variability is not surprising given the crudeness of the squeezing mechanism. (The figure shows internal evidence that it cannot be the case that the extra dots on the S plate are from some slight overlap from the mcherry signal into the FITC range. The extra dots are at the high end of the FITC signal range not the low or barely detectable part of the range.)

Table 1 reinforces the observation that the squeezed cells, plates O and S, have more GFP expressing cells and especially more higher expressing cell than does the plate which had the non-squeezed cells, plate N. It also shows that a less than 2-fold increase in the number of GFP positive cells, i.e. 1.8 fold, resulted in a more than 9-fold increase in the number of GFP expressing vectors. This reinforces the claim that when the squeezing promotes transduction in a cell it does so in a manner that promotes multiple expressing vectors in that cell. Table 2 compares the numbers of cells at the highest expressing levels showing that the squeezing procedure results in more cells with a higher number of GFP expressing vectors. Comparing the number of cells in the three highest expressing levels in each plate. N has 8 cells. O. has 182 cells. S has 486 cells. Again, Plate S is showing a higher numbers of GFP positive cells and a higher percentage of GFP positive cells than plate O because it just happened that in this case the crude method of squeezing the cells gave more cells with the desired degree of squeezing to that population.

11 of the cells with the strongest GFP expression were identified before harvest. Of these 11 chosen cells, chosen because they were expressing GFP strongly, 9 were also visibly expressing mCherry when the filters were switched. 2 of the 11 cells were expressing only GFP. The expectation, if infection and subsequent expression were random is that only 1% of the GFP expressing cells would also express mCherry because only about 1% of the cells on that plate expressed mCherry after infection with that vector. In unrelated assays with AAV2-TRUF11 infection of hep G2 cells done without squeezing it was observed that infection was random and seemed not limited to some subset of cells. The correlation in those assays between amount of vector and number of cells converted to FITC expression was linear up to about 20-30% of the cells becoming positive. At this point the graph becomes lightly less than linear consistent with the likelihood that some cells now had acquired 2 expressing vectors. If this sort of assay is carried to the limit, at most only 10-20% of the cells in the whole population seem poorly infectable at any point. This assay with the 11 chosen cells implies that in some subpopulation of infected cells the likelihood of one expressing vector per cell at low levels of vector infection breaks down. (As noted above only 1% of the cells are expressing mCherry.) In this subpopulation there are clearly more than one vector per cell, based on both the brightness of these cells and more definitively on the presence of the m Cherry vector and the TRUF1 vector in the same cells. The presence of both vectors in the same cell implies it is not the case that the extra brightness of some cells is explained by amplification of the TRUF11 genome in that subpopulation.

FIGS. 9 and 10 and 11 show example of the same field of view from a plate infected with both AAV2-TRUF11 and AAV-mCherry. The plate is confluent at the time of photography. The field of view was photographed with a TRUF11 filter, then again with an mCherry filter. Some cells that are expressing both vectors at levels detectable in this micrograph are marked with numbers. The same number in both the red and green fields indicates cells in the same position. As seen some cells are expressing both genes. Some cells with both vectors were left unmarked to make the slide simpler. (In numerous previous assays Hep G 2 cells were infected with both the GFP and mCherry vectors, without squeezing the cells. Only one cell was ever seen that expressed both GFP and mCherry.

It is expected that many cells that are expressing vector will express only one vector because many transductions are occurring in cells that have did not have vector entering the nucleus through a ruptured nuclear membrane.

The requirements for vector entry by nuclear rupture are stringent: A ruptured nuclear membrane, no or minimal rupture of the cellular membrane. In the crude method of squeezing employed in these assays it is likely that most cells were either squeezed too little or squeezed too much.

These assays advance several conclusions: 1. Squeezing of preinfected cells results in more cells with vector expression. 2. Squeezing of preinfected cells results in faster expression of the transgene carried by the vector. 3. Squeezing of preinfected cells results in cells that express more than one vector and this population of cells that are expressing more than one vector is a substantial fraction of the expressing cells.

### Uses of this Technology

As described this technology has 2 components. The first is entrance of the rAAV vector into the cell nucleus by induced nuclear membrane breakage. The second component is that with nuclear membrane breakage it should be possible to use much lower levels of vector. This second aspect can be used independently of induced breakage. Described below is use with induced breakage then use with or without induced breakage is described where the advantageous novelty is the possibility of doing gene therapy with much reduced amounts of vector.

This method of infection with induced nuclear membrane breakage will need to be performed on cells ex vivo. One advantage is that to reach any predetermined level of transduction, less vector will be required than for transduction by normal nuclear entry.

The method provides two additional advantages for gene therapy with rAAV vectors and likely with other viral vectors in which the vector particles accumulate at the nuclear membrane.

As shown with this method even with low levels of successfully transduced cells, those cells that are successfully transduced, are often transduced by more than one vector. This second advantage is in contrast to standard vector infection in which very few cells acquire more than one vector. With more than one vector a cell will express the transgene at higher levels. If infection is by vectors carrying different transgenes this permits the creation of cells with functioning vector genomes of more than one transgene sequence.

Another, more substantial usefulness to this second advantage is as follows: The rAAV particle is limited with respect to the length of the genome it can contain, which is approximately 4.5 Kb. The genomes of certain genes with promise in gene therapy, cystic fibrosis being a prominent example, are too large to fit into the capsid. This has forced clinicians to perform gene therapy with a truncated genome, in the case of cystic fibrosis with what are designated as the mini and the micro genome. These genomes encompass the most important regions of the cystic fibrosis genome, but the complete genome would undoubtedly provide a better therapeutic effect. A goal in rAAV gene therapy has been to enable the infection of cells with more than one vector genome to enable recombination that will reassemble the whole of the therapeutic gene.

(Recombination between different rAAV vectors in a dually infected target cell has been found to be relatively efficient.) This method offers a way to put more than one vector into the target cell thereby making possible recombination within the targe cell to create full-length genomes.

The third advantage is rapid expression of the transgene. This is of importance when considering the possibility of combatting certain infectious diseases or toxic agents such as snake venom. Normal vaccination in which an antigen is given to a patient requires approximately two weeks for the production of antibodies. A more rapid method of antibody production is gene therapy in which the transgene codes for an antibody or immunoadhesin rather than an antigen. This is an antibody previously shown to be effective against the infectious or toxic agent. Preselection and direct production of the immunoadhesin bypasses the normal role of the immune system in antibody production. This combination of preselection and use of recombinant AAV vectors to deliver these immunoadhesins or antibodies has most dramatically been shown effective against HIV infection. (18) (19) (20). In an appealing modification the delivered transgene can code for nanobodies. The method described herein can further reduce the time between infection with a vector and production of an immunoadhesin.

### Induced Rupture of the Nuclear Membrane

### Rabies

Rabies is easily treatable by post exposure vaccination. Despite this, an estimated 50,000 people die of rabies each year, though this estimate may be substantially lower than the actual number of yearly deaths since rabies is most prevalent in regions with suboptimal health monitoring. This high number of deaths is due a failure to deliver post exposure prophylaxis. There are several reasons for this but the basic reason is the expense of rabies vaccine and anti-rabies immunoglobulin. When someone is bitten in the parts of the world where rabies in dogs is prevalent, (on a world-wide basis 99% of human rabies is due to bites from domesticated dogs) it is often not known whether the dog was rabid. In addition, bites from rabid dogs usually do not result in the victim developing rabies, particularly if the bite was far from the head. Given these two sources of uncertainty, the high cost of vaccination, and the even higher cost of immunoglobulin, many victims are not treated. If symptoms do develop, it is of course too late to prevent progress of the disease. A lower cost vaccine or immunoglobulin treatment might enable a larger fraction of these potentially infected persons to receive treatment. An injection of rAAV vectors coding for an anti-rabies nanobody given by the method described herein could provide the production of these nanobodies in a reasonably timely manner. Like the use of immunoglobulin this method has the promise of delivering immunoadhesins to the victim's circulation more rapidly than vaccination with a rabies antigen.

An immediate objection to this scenario is the present high cost of rAAV gene therapy. This high cost, however, is due to the effort to recover the high expenses incurred for development and clinical trials for diseases that have few patients. There is little reason intrinsic to the manufacturing of these vectors for this high cost. In addition the method described herein holds the promise of being effective at much lower doses of vector than is required for standard gene therapy. A necessary corollary is the development of a system for local storage of the vector. This method can be used for other infectious diseases, particularly for disease outbreaks.

### Proposed Method for Delivering Vector Through Nuclear Membrane Rupture

In the cases of a potential exposure to rabies or another infectious agent and snake bite venoms, (described below) the procedures to deliver vector can be as follows: Blood is withdrawn from the patient. Then either the rAAV can be added either to the whole withdrawn blood or the blood can be given a standard spin to separate plasma from red blood cells and from non-red blood cells with the latter being given the rAAV vector. The blood virus mixture is incubated a short time. Then it is returned to the patient after it has passed through a filter that is a matrix or a membrane containing pores of the appropriate dimensions to accomplish nuclear membrane rupture without cell membrane rupture. The passage through the membrane and redelivery to the patient can be accomplished in one step by creating a dedicated syringes containing a membrane with the appropriate pore sizes for reinjection into the patient. (FIG. 12).

Most of the cells in peripheral blood are red blood cells and thrombocytes. Leukocytes comprise only about 1 percent of the peripheral blood cell population. Of these only a small fraction are B cells, the ideal target for gene therapy. Since the goal of this approach is rapid protein expression, isolation of specific cell types and ex vivo expansion would be counterproductive. It remains an unanswered question whether this method of infection would give useful levels of transduced cells among the unamplified granulocyte, T-cell, N-K cell and monocyte populations.

In a hospital setting an alternative method of squeezing can be employed that would be useful in standard gene therapy. In this setting cells of more diverse types can be squeezed i.e. cells of the many types that are being considered for ex vivo gene therapy. Cells can be removed from the patient and separated into individual cells for tissue culture. These cells can be amplified if desired. Squeezing can be more systematically and thoroughly accomplished by forcing media containing cells through individual orifices of a solid material, e.g. glass or metal, in which the orifice or multiple orifices can have a consistent dimension, and the cells need only pass through an orifice once or a few times. The advantage of using this procedure in ex vivo gene therapy is an ability to transduce most of the cells and to transduce with a smaller amount of vector.

### Anti Snake Bite Venom.

Another potential use for a rapid method of delivering transduced cells to the blood is the treatment of individuals bitten by venomous snakes. On a world-wide basis an estimated 100,000 persons a year die from snake bites, mostly in parts of Africa and Asia that are less well served by a medical infrastructure. (WHO data.). Approximately three times this number suffer from long-term effects of the venom. The principal treatments for snake bite victims are antivenoms which are relatively crude mixtures of antibodies to the venom. Antivenoms are generally raised by immunization of horses. As such, antivenoms themselves can cause significant immunological problems in patients. In addition, the immunoglobulin preparations are expensive and often unavailable where they are most needed. A further complication is that in areas of the world where snake bites are a serious problem there are numerous species of poisonous snakes. The venoms of most of these snakes is a mixture of many different molecules, as many as 100 per species, many of which are toxic. About 90% of the dry weight of venoms is protein.

In a recent landmark study from the Laustsen laboratory at The Technical University of Denmark, that built on decades of work by themselves and other groups, it was shown possible to identify broadly neutralizing, high affinity nanobodies against snake venoms. (21). They were able to identify nanobodies that would cross react with multiple toxins. A mixture of 8 of these nanobodies protected against death and markedly reduced venom-induced tissue damage for 17 African elapid snakes. This work represents a significant leap forward. The use of nanobodies made in tissue culture will reduce the side effects seen with animal derived immunoglobulins. The important point is that with one mixture these workers were able to accomplish resistance against a long list of snake venoms without needing to identify which species of snake was responsible. This work was directed against venom from African snakes, but it is likely that similar libraries of nanobody antivenoms could be created for the snakes prevalent in other regions such as South and Southeast Asia.

This work raises the possibility of using AAV based gene therapy as a snake bite therapy. The use of nanobodies rather than antibodies permits a consideration of using a mix of rAAV vectors since the needed coding capacity in the vector is greatly reduced. Even more importantly the ability to furnish, with a library of preselected nanobodies, protection against hundreds of toxins (the number of different toxins found in 17 different snake species) allows the use of fewer rAAV molecules in a therapeutic mix. Genomes coding for 8 nanobodies can be contained within 3 rAAV particles. The principal advantages of using a mix of rAAV particles versus a mix of nanobody proteins are twofold. First, the former is easier to purify and easier to store and should therefore be less expensive to produce and in theory at least should reduce the problem of availability. Second, since in the former the production of nanobody is within the patient and continuous, the problem of the short half-life of nanobodies in circulation is reduced. (The half-life of standard immunoglobulin antibodies in patients ranges from 24-96 hrs. The half-life of nanobodies in patients is thought to be much less.)

The principal drawback of the former is that the time between injection into the patient and nanobody activity is longer than the time between injection of immunoglobulin and activity since the nanobodies need to be produced from the genomes carried by the rAAV particles. One of the aims of the methodology described herein is to reduce that time difference. The 17 snake species investigated by Ahmadi et al (21) were from the elapid family, which includes, cobras, mambas, kraits and other species. If an elapid bite is lethal, death is usually within several hours. Most snake bite victims survive however, though often severely injured by the venom. Non-lethal damage by the components of the venom can be ongoing long after the snake bite in part because venom is frequently not totally absorbed into target tissues until long after the bite. Consequently, if the patient has survived and an antivenom is available, the sooner the better but it is almost never too late to be of some benefit. This delay between bite and full effect of the venom highlights a usefulness of a gene therapy approach; fresh antivenom is being produced continually by the patient for some time after delivery of the vector. This is even more relevant if the anti-venom is a nanobody rather than an antibody, due to the shorter serum half-life of the former.

Other potential use of this technology are against viral infections other than rabies, e.g. Ebola virus. When someone has been infected with a virus or is at immediate risk of infection, in many cases an antibody to the virus is needed more rapidly than is the case with a rabies infection. Immunoglobulins would be an obvious candidate, but, as mentioned above, immunoglobulins are prohibitively expensive. In addition, in some cases, Ebola being an example, immunoglobulins produced in horses have been somewhat ineffective. An antibody or nanobody genome that was specifically tailored for human use would likely overcome this limitation. The advantages of using the methodology of this application on patients likely to have been or in the future to be infected with a viral pathogen are the same as described above for rabies.

### Endogenous Nuclear Membrane Rupture.

The above description relates to doing gene therapy by inducing a breakage of the nuclear membrane. However, breakage of the nuclear membrane can also be the result of endogenous processes. Nuclear membrane breakage is intrinsic to several disease conditions. Some of the procedures described above that would be employed with induced breakage also can be employed for several of the conditions in which the breakage was endogenous. One class of diseases are those produce by mutations in the genes coding for the nuclear lamina. Collectively these conditions are referred to as the laminopathies. Approximately 15 diseases have been described in which the cause is a mutation in the most prominent Lamina, Laminin A. This makes Laminin A the human gene with the most known disease causing mutations. (22). Additionally, there are mutations in other lamina and in the nuclear pore complex that affect the integrity of the nuclear membrane in a manner that renders rupture of the membrane more common. These diseases include several of the muscular dystrophies, lipodystrophies and progeroid syndromes. Many of these conditions could likely be ameliorated by delivering appropriate genes to the affected cells. Obstacles to the development of gene therapies for these conditions are the large number of different conditions and the rarity of most.

This application describes a gene therapy approach for many of these conditions. Entry of AAV vector particles into the cell and migration of these particles to the nucleus is apparently non-specific. Much of the specificity in infection by rAAV vectors seems to be at entrance to the nucleus. However, with a broken nuclear membrane this specificity is no longer of relevance. This lack of specificity in admission to the nucleus after nuclear membrane disruption is demonstrated by the appearance of cytoplasmic proteins and organelles in the nucleus after rupture. (23). It seems likely that most rAAV particles produced by current methods are defective in their ability to enter the nucleus, which is why, though many particles cluster around the nucleus of rAAV infected cells few gain entrance leading to levels of transduction that are much lower than the number of vector particles delivered. (13). In this last cited report, Zeltner at. al., AAV particles, i.e. the virus not the vector derived from the virus, were shown to have almost perfect infectivity presumably because they, unlike the vector particles were well made. Vector particles that were heretofore deficient in their ability to gain entrance to the nucleus, i.e. most particles, would now seem on a par with the few vector particles that could heretofore gain entrance presumably because the latter particles were well made. This facilitated entrance is because with a ruptured nuclear membrane nuclear entrance is now passive i.e. not requiring any function by the particle, i.e. not requiring that they be so well made. This should allow therapy to be accomplished with much fewer particles delivered to the cells.

A method of treating such patients is to deliver to the patient very low levels of vector, which, after entering a cell, would, as described above migrate to the periphery of the nucleus. Although it would be desirable it is not an absolute necessity to achieve rapid expression of a corrective transgene in many of these conditions, so a long interval between infection with the vector and first expression of the transgene is not an insurmountable obstacle. With repeated vector administrations a population of mutant cells can, through intermittent nuclear membrane rupture, gradually transition to one carrying a corrective transgene in many of its cells. This transgene may have a paracrine effect, if desired. The very low level of vector given both on the first infection and on subsequent infections is designed to minimize adaptive and innate host immune responses.

In sum, this methodology will reduce obstacles to a gene therapy treatment. The first is that in this method it is a manifestation of the disease, i.e. nuclear membrane rupture, that provides the targeting mechanism for the vector. The vector, though delivered systemically, will express the transgene preferentially in the appropriate cells without a requirement to tailor a vector's capsid to the cell type, though a capsid directed at a designated cell type would provide an additional level of specificity. The second, as mentioned, is that this method of targeting can reduce the needed quantities of vector particles.

With this method nuclei are likely to take up more than one vector particle. Advantages of this are, as described above, the possibility of recombination between two vectors each carrying different sequences of the same large gene to create full-length proteins. A second advantage is that the wt AAV genome could be delivered along with the vector carrying the recombinant genome. (24) (16). This would permit Rep-dependent integration of the recombinant genome into the safe harbor AAVS1 site (25). Integration is necessary if the targeted cell will undergo subsequent rounds of cell division.

An additional condition in which this approach might be useful is cancer. Some cancers can be characterized by having frequent blebbing, of the nuclear membrane leading to rupture of this membrane (23). For many genes potentially useful in treating cancer, expression in non-cancer cells would be undesirable. Examples would be genes that might be fatal to the cell that expresses them, or that would block further cell division, or that might provoke a local immune response. The advantage of this method is that since cancer cells have more frequent nuclear membrane ruptures than non-cancer cells this method offers another level of specificity in the targeting of cancer cells.

A common source of nuclear membrane disruption in cancer cells is thought to be metastasis. The ability of cancer cells to migrate is limited by the size of the nucleus (26). The ability of cancer cells to metastasize is dependent on nuclear deformation. (27) Cancer cells when migrating through small spaces in model systems undergo nuclear membrane rupture. (28) The greater the degree of cell confinement the greater the amount of nuclear membrane rupture. This mirrors the squeezing effects on the cell when they are metastasizing and results in some exchange of nuclear and cytoplasmic elements. This exchange causes damage to the metastasizing cell's genome due to contact with cytoplasmic elements and so it is likely that metastasis that involves nuclear membrane rupture increases the heterogeneity and therefore the potential harmfulness of the metastatic population. (28). (17). Nuclear membrane rupture during metastasis, in addition to enabling the metastatic movement, has been shown to enhance tumor progression by effects on the metastasizing cell (29).

As in the example described above, rAAV gene therapy that targeted metastasizing cancer cells could be performed at low multiplicities of infection. In addition, it is better able to deliver more than one vector particle into the cell. This is particularly useful for treating cancer cells because with a combined delivery of wt AAV and rAAV vectors, it is likely that many of the metastasizing cells will acquire both types of particles. This will allow integration of the rAAV genome into the genome of the metastasizing cells as promoted by the Rep protein coded by the wt AAV. (25) In a coinfected cancer cell line, an rAAV vector and a wt AAV virus, most of the integrated rAAV genomes integrated into the AAVS1 region indicating that the integration had been supported by the AAV Rep protein. (16)

Since these are dividing cells, having an integrated transgene is necessary if the goal is to have transgene expression in progeny cells for multiple cell divisions. In addition, this scenario points to a target for an anti-metastasis gene therapy. Survival of cells that have a ruptured nuclear membrane requires that the cell maintain an ability to repair this membrane efficiently to prevent excessive intermixing of nuclear and cytoplasmic contents (28). Gene therapy that delivered genes that inhibited nuclear membrane repair might be a way to specifically eliminate metastasizing cells.

### REFERENCES

1. G. Seisenberger et al., Real-time single-molecule imaging of the infection pathway of an adeno-associated virus. Science 294, 1929-1932 (2001).
2. P. J. Xiao, R. J. Samulski, Cytoplasmic trafficking, endosomal escape, and perinuclear accumulation of adeno-associated virus type 2 particles are facilitated by microtubule network. J Virol 86, 10462-10473 (2012).
3. J. S. Bartlett, R. Wilcher, R. J. Samulski, Infectious entry pathway of adeno-associated virus and adeno-associated virus vectors. J Virol 74, 2777-2785 (2000).
4. W. Xiao, K. H. Warrington, Jr., P. Hearing, J. Hughes, N. Muzyczka, Adenovirus-facilitated nuclear translocation of adeno-associated virus type 2. J Virol 76, 11505-11517 (2002).
5. K. Lux et al., Green fluorescent protein-tagged adeno-associated virus particles allow the study of cytosolic and nuclear trafficking. J Virol 79, 11776-11787 (2005).
6. C. E. Thomas, T. A. Storm, Z. Huang, M. A. Kay, Rapid uncoating of vector genomes is the key to efficient liver transduction with pseudotyped adeno-associated virus vectors. J Virol 78, 3110-3122 (2004).
7. F. Sonntag, S. Bleker, B. Leuchs, R. Fischer, J. A. Kleinschmidt, Adeno-associated virus type 2 capsids with externalized VP1/VP2 trafficking domains are generated prior to passage through the cytoplasm and are maintained until uncoating occurs in the nucleus. J Virol 80, 11040-11054 (2006).
8. S. C. Nicolson, R. J. Samulski, Recombinant adeno-associated virus utilizes host cell nuclear import machinery to enter the nucleus. J Virol 88, 4132-4144 (2014).
9. J. M. Kelich et al., Super-resolution imaging of nuclear import of adeno-associated virus in live cells. Mol Ther Methods Clin Dev 2, 15047 (2015).
10 S. Cohen, A. K. Marr, P. Garcin, N. Panté, Nuclear envelope disruption involving host caspases plays a role in the parvovirus replication cycle. J Virol 85, 4863-4874 (2011).
11. M. Porwal et al., Parvoviruses cause nuclear envelope breakdown by activating key enzymes of mitosis. PLOS Pathog 9, e1003671 (2013).
12. A. Rossi et al., Vector uncoating limits adeno-associated viral vector-mediated transduction of human dendritic cells and vector immunogenicity. Sci Rep 9, 3631 (2019).
13 N. Zeltner, E. Kohlbrenner, N. Clement, T. Weber, R. M. Linden, Near-perfect infectivity of wild-type AAV as benchmark for infectivity of recombinant AAV vectors. Gene Ther 17, 872-879 (2010).
14. J. C. Grieger, S. M. Soltys, R. J. Samulski, Production of Recombinant Adeno-associated Virus Vectors Using Suspension HEK293 Cells and Continuous Harvest of Vector From the Culture Media for GMP FIX and FLT1 Clinical Vector. Mol Ther 24, 287-297 (2016).
15. C. A. Laughlin, J. D. Tratschin, H. Coon, B. J. Carter, Cloning of infectious adeno-associated virus genomes in bacterial plasmids. Gene 23, 65-73 (1983).
16. P. Ward, C. E. Walsh, Targeted integration of a rAAV vector into the AAVS1 region. Virology 433, 356-366 (2012).
17. M. Raab et al., ESCRT III repairs nuclear envelope ruptures during cell migration to limit DNA damage and cell death. Science 352, 359-362 (2016).
18. P. R. Johnson et al., Vector-mediated gene transfer engenders long-lived neutralizing activity and protection against SIV infection in monkeys. Nat Med 15, 901-906 (2009).
19. B. C. Schnepp, P. R. Johnson, Adeno-associated virus delivery of broadly neutralizing antibodies. Curr Opin HIV AIDS 9, 250-256 (2014).
20. A. B. Balazs et al., Antibody-based protection against HIV infection by vectored immunoprophylaxis. Nature 481, 81-84 (2011).
21. S. Ahmadi et al., Nanobody-based recombinant antivenom for cobra, mamba and rinkhals bites. Nature 647, 716-725 (2025).
22. K. H. Schreiber, B. K. Kennedy, When lamins go bad: nuclear structure and disease. Cell 152, 1365-1375 (2013).
23 J. D. Vargas, E. M. Hatch, D. J. Anderson, M. W. Hetzer, Transient nuclear envelope rupturing during interphase in human cancer cells. Nucleus 3, 88-100 (2012).
24. C. Zhang, N. G. Cortez, K. I. Berns, Characterization of a bipartite recombinant adeno-associated viral vector for site-specific integration. Hum Gene Ther 18, 787-797 (2007).
25. R. M. Kotin et al., Site-specific integration by adeno-associated virus. Proc Natl Acad Sci USA 87, 2211-2215 (1990).
26 K. Wolf et al., Physical limits of cell migration: control by ECM space and nuclear deformation and tuning by proteolysis and traction force. J Cell Biol 201, 1069-1084 (2013).
27. Y. Fu, L. K. Chin, T. Bourouina, A. Q. Liu, A. M. VanDongen, Nuclear deformation during breast cancer cell transmigration. Lab Chip 12, 3774-3778 (2012).
28. C. M. Denais et al., Nuclear envelope rupture and repair during cancer cell migration. Science 352, 353-358 (2016).
29. B. R. Gauthier, P. I. Lorenzo, V. Comaills, Physical Forces and Transient Nuclear Envelope Rupture during Metastasis: The Key for Success? Cancers 14, 83 (2022).

1. An rAAV gene therapy method that results in an increased number of expressing recombinant adeno-associated virus vectors in cells infected with said vector and in a decreased length of time between infection of the cell by the vector and expression of the transgene in comparison with standard methods of infection, which method comprises: infecting the cells; incubating the cells until vector has accumulated around the nuclear envelope; squeezing the infected cells individually to an extent that is mild enough to leave the cell membrane unruptured, so that the cells survive, but that is sufficiently robust to rupture the nuclear membrane so that vector particles enter the nucleus in a passive manner.

2. The method of claim 1 in which squeezing the cells is accomplished by forcing or allowing the cells to pass through small openings in a matrix or membrane or filter with pores of the requisites size for rupturing the nuclear membrane while leaving the cellular membrane unruptured.

3. The method of claim 1 in which transduction is accomplished with many fewer vector particles per targeted cell than is possible with standard infection due to lack of a need for efficient nuclear entry functions on the vector particle and to multiple particles entering a cell.

4. The method of claim 1 in which a patient's blood is withdrawn, infected with the vector, incubated, then passed through a matrix or membrane or filter of appropriately sized passages and redelivered to the patient.

5. The method of claim 1 in which a patient's blood is withdrawn, infected with the vector, incubated, then passed through a matrix or membrane or filter of appropriately sized passages and redelivered to the patient, which redelivery is accomplished by a syringe like device in which the patients cells are squeezed through a matrix or membrane or filter as the cells are being injected into the patient.

6. The method of claim 1 whereby infection of a patient is with a rAAV vector carrying a transgene that encodes a preselected anti rabies or anti other viral nanobody or antibody.

7. The method of claim 1 whereby infection of a patient is with rAAV vectors carrying transgenes that encode preselected anti snake venom nanobodies or antibodies.

8. A method of gene therapy with rAAV vectors in which cells to be targeted have endogenous nuclear membrane rupture; which method can consist of infections at very low levels of vector so as to provoke a minimal adaptive or innate immune response, which low vector level is made feasible because the passive uptake allows productive therapy by particles that would not be able to gain nuclear entry by an active mechanism; followed by multiple rounds of low level infections separated by intervals of waiting.

9. The method of claim 8 in which the cells to be targeted for the rAAV therapy have a genetic defect one of whose effects is an abnormally high levels of nuclear membrane rupture in some cells in which nuclear membrane rupture serves in turn both as a targeting signal for the vector and a mechanism for vector entry into the nucleus.

10. The method of claim 8 in which the diseases to be targeted are one of the nuclear laminopathies that cause a number of muscular dystrophies, lipodystophies and progeroid syndromes.

11. The method of claim 8 in which the cells to be targeted for the rAAV therapy have disease condition one of whose effects is an abnormally high levels of nuclear membrane rupture which in turn serves as a targeting signal for the vector.

12. The method of claim 8 in which the cells to be targeted for the rAAV therapy are metastasizing cancer cells in which squeezing through tight cellular junctions causes nuclear membrane rupture allowing vector entry into the nucleus and which rupture also serves as a targeting signal for the vector.

13. The method of claim 8 whereby infection of a patient is with a rAAV vector and a wt virus simultaneously, allowing most cells that are transduced by the rAAV to also be transduced by the wt AAV in order to enable wt AAV support of integration of the rAAV vector genome into the genome of the infected cell.

14. The method of claim 8 in which passive entry allows uptake by a nucleus of multiple vector particles including particles containing different transgene sequences from the same gene or different genes that will in the former case allow recombination between the sequences to create a construct that will code for a more complete protein.
